(19) United States
(12) Patent Application Publication
(10) Pub. No.: US 2022/0241305 A1
(43) Pub. Date: Aug. 4, 2022

(54) ETHYL CELLULOSE BASED COATINGS FOR MICROENCAPSULATION OF NICOTINAMIDE RIBOSIDE, NICOTINIC ACID RIBOSIDE, REDUCED NICOTINYL RIBOSIDE COMPOUNDS, AND NICOTINYL RIBOSIDE COMPOUND DERIVATIVES

(71) Applicant: ChromaDex Inc., Irvine, CA (US)

(72) Inventors: Aron Erickson, Longmont, CO (US); Amanda Storjohann, Westminster, CO (US); Joshua Holloway, Longmont, CO (US); Philip Redpath, Portadown (GB)

(73) Assignee: ChromaDex Inc., Irvine, CA (US)

(21) Appl. No.: 17/589,422

(22) Filed: Jan. 31, 2022

Related U.S. Application Data
(60) Provisional application No. 63/143,346, filed on Jan. 29, 2021.

(51) Int. Cl. A61K 31/706; A61K 9/50

(52) U.S. Cl. CPC A61K 31/706; A61K 9/5015; A61K 9/5047; A61K 9/5089

(57) ABSTRACT

A process is provided for microencapsulating nicotinamide riboside (NR), and other NR derivatives by using ethyl cellulose which may be polymerized in a particular manner. Further, compositions comprising NR or derivatives thereof microencapsulated in an edible biopolymer are provided.

This application claims the benefit of U.S. Provisional appl. No. 63/143,346, filed on Jan. 29, 2021, which is hereby incorporated by reference in its entirety.

### TECHNICAL FIELD

The present invention relates to a process for microencapsulating nicotinamide riboside (NR), and other nicotinyl riboside compounds which are NAD+ precursors. NR, although hydrophilic, is particularly susceptible to hydrolysis, such that microencapsulation using an edible and biocompatible polymer provides physicochemical stability.

### BACKGROUND

Nicotinamide riboside (NR) is a valuable bioactive intermediate. This compound has been implicated in processing and metabolic pathways involving NAD+(J. Priess and P. Handler, J. Biol. Chem. (1958) 233:488-492). Nicotinic acid and nicotinamide, collectively niacins, are the vitamin forms of nicotinamide adenine dinucleotide (NAD+). Eukaryotes can synthesize NAD+de novo via the kynurenine pathway from tryptophan (Krehl, et al. Science (1945) 101:489-490; Schutz and Feigelson, J. Biol. Chem. (1972) 247:5327-5332) and niacin supplementation prevents the pellagra that can occur in populations with a tryptophan-poor diet. Thus, it is well-established that nicotinic acid is phosphoribosylated to nicotinic acid mononucleotide (NaMN), which is then adenylylated to form nicotinic acid adenine dinucleotide (NaAD), which in turn is amidated to form NAD+(Preiss and Handler (1958) 233:488-492; Ibid., 493-50).

Nicotinamide Adenine Dinucleotide (“NAD⁺”) is an enzyme co-factor that is essential for the function of several enzymes related to reduction-oxidation reactions and energy metabolism. (Katrina L. Bogan & Charles Brenner, Nicotinic Acid, Nicotinamide, and Nicotinamide Riboside: A Molecular Evaluation of NAD⁺ Precursor Vitamins in Human Nutritions, 28 Annual Review of Nutrition 115 (2008)). NAD⁺ functions as an electron carrier in cell metabolism of amino acids, fatty acids, and carbohydrates. (Bogan & Brenner 2008). NAD⁺ serves as an activator and substrate for sirtuins, a family of protein deacetylases that have been implicated in metabolic function and extended lifespan in lower organisms. (Laurent Mouchiroud et al., The NAD⁺/Sirtuin Pathway Modulates Longevity through Activation of Mitochondrial UPR and FOXO Signaling, 154 Cell 430 (2013)). The co-enzymatic activity of NAD⁺, together with the tight regulation of its biosynthesis and bioavailability, makes it an important metabolic monitoring system that is clearly involved in the aging process.

Once converted intracellularly to NAD(P)⁺, vitamin B3 is used as a co-substrate in two types of intracellular modifications, which control numerous essential signaling events (adenosine diphosphate ribosylation and deacetylation), and is a cofactor for over 400 reduction-oxidation enzymes, thus controlling metabolism. This is demonstrated by a range of metabolic endpoints including the deacetylation of key regulatory proteins, increased mitochondrial activity, and oxygen consumption. Critically, the NAD(P)(H)-cofactor family can promote mitochondrial dysfunction and cellular impairment if present in sub-optimal intracellular concentrations. Vitamin B3 deficiency yields to evidenced compromised cellular activity through NAD⁺ depletion, and the beneficial effect of additional NAD⁺ bioavailability through nicotinic acid (“NA”), nicotinamide (“Nam”), and nicotinamide riboside (“NR”) supplementation is primarily observed in cells and tissues where metabolism and mitochondrial function had been compromised. Interestingly, supplementation with nicotinic acid (“NA”) and nicotinamide (“Nam”), while critical in acute vitamin B3 deficiency, does not demonstrate the same physiological outcomes compared with that of nicotinamide riboside (“NR”) supplementation, even though at the cellular level, all three metabolites are responsible for NAD⁺ biosynthesis. This emphasizes the complexity of the pharmacokinetics and bio-distribution of B3-vitamin components.

The bulk of intracellular NAD⁺ is believed to be regenerated via the effective salvage of nicotinamide (“Nam”) while de novo NAD⁺ is obtained from tryptophan. (Anthony Rongvaux et al., Reconstructing eukaryotic NAD metabolism, 25 BioEssays 683 (2003)). Crucially, these salvage and de novo pathways apparently depend on the functional forms of vitamins B1, B2, and B6 to generate NAD⁺ via a phosphoriboside pyrophosphate intermediate. Nicotinamide riboside (“NR”) is the only form of vitamin B3 from which NAD⁺ can be generated in a manner independent of vitamins B1, B2, and B6, and the salvage pathway using nicotinamide riboside (“NR”) for the production of NAD⁺ is expressed in most eukaryotes.

The main NAD⁺ precursors that feed the salvage pathways are nicotinamide (“Nam”) and nicotinamide riboside (“NR”). (Bogan & Brenner 2008). Studies have shown that nicotinamide riboside (“NR”) is used in a conserved salvage pathway that leads to NAD⁺ synthesis through the formation of nicotinamide mononucleotide (“NMN”). Upon entry into the cell, nicotinamide riboside (“NR”) is phosphorylated by the NR kinases (“NRKs”), generating NMN, which is then converted to NAD⁺ by nicotinamide mononucleotide adenylyltransferase (“NMNAT”). (Bogan & Brenner 2008). Because NMN is the only metabolite that can be converted to NAD⁺ in mitochondria, nicotinamide (“Nam”) and nicotinamide riboside (“NR”) are the two candidate NAD⁺ precursors that can replenish NAD⁺ and thus improve mitochondrial fuel oxidation. A key difference is that nicotinamide riboside (“NR”) has a direct two-step pathway to NAD⁺ synthesis that bypasses the rate-limiting step of the salvage pathway, nicotinamide phosphoribosyltransferase (“NAMPT”). Nicotinamide (“Nam”) requires NAMPT activity to produce NAD⁺. This reinforces the fact that nicotinamide riboside (“NR”) is a very effective NAD⁺ precursor. Conversely, deficiency in dietary NAD⁺ precursors and/or tryptophan causes pellagra, a disease characterized by dermatitis, diarrhea, and dementia. (Bogan & Brenner 2008).

In summary, NAD⁺ is required for normal mitochondrial function, and because mitochondria are the powerhouses of the cell, NAD⁺ is required for energy production within cells. NAD+ was initially characterized as a co-enzyme for oxidoreductases. Though conversions between NAD+, NADH, NADP and NADPH would not be accompanied by a loss of total co-enzyme, it was discovered that NAD+ is also turned over in cells for unknown purposes (Maayan, Nature (1964) 204:1169-1170). Sirtuin enzymes such as Sir2 of S. cerevisiae and its homologs deacetylate lysine residues with consumption of an equivalent of NAD+ and this activity is required for Sir2 function as a transcriptional silencer (Imai, et al., Cold Spring Harb. Symp. Quant. Biol. (2000) 65:297-302). NAD+-dependent deacetylation reactions are required not only for alterations in gene expression but also for repression of ribosomal DNA recombination and extension of lifespan in response to calorie restriction (Lin, et al., Science (2000) 289:2126-2128; Lin, et al., Nature (2002) 418:344-348). NAD+ is consumed by Sir2 to produce a mixture of 2′- and 3′ O-acetylated ADP-ribose plus nicotinamide and the deacetylated polypeptide (Sauve, et al., Biochemistry (2001) 40:15456-15463). Additional enzymes, including poly(ADPribose) polymerases and cADPribose synthases are also NAD+-dependent and produce nicotinamide and ADPribosyl products (Ziegler, Eur. J. Biochem. (2000) 267:1550-1564; Burkle, Bioessays (2001) 23:795-806).

The non-coenzymatic properties of NAD+ has renewed interest in NAD+ biosynthesis. FIG. 1 describes how NAR, NR and other metabolic intermediates are transformed to NAD+. In short, the biosynthetic pathway for NAR proceeds directly to NaMN, then NaAD, and ultimately to form NAD+.

If NR, or its derivatives, salts, or prodrugs thereof, as described herein, could be developed in a more protected form, such as a microencapsulated form, this would confer stability and better handling. A microencapsulated NR, or its derivatives, salts, or prodrugs thereof, would be useful in pharmaceuticals, food or beverages, or dietary supplements, e.g., to enhance NAD+ levels in cells, which would represent a useful contribution to the art.

### SUMMARY

A composition comprises NR, or a salt or solvate thereof, microencapsulated in a biopolymer shell.

In one embodiment, the composition comprises polymerized ethyl cellulose encapsulating nicotinamide riboside chloride salt. The polymerized ethyl cellulose may include 10% by weight additional nicotinamide riboside chloride. In an alternative embodiment, the composition comprises polymerized ethyl cellulose encapsulating a different salt of nicotinamide riboside, as described herein.

In another embodiment, the composition comprising polymerized ethyl cellulose encapsulating nicotinamide riboside chloride salt, the polymerized ethyl cellulose may include 10% by weight additional nicotinamide riboside chloride, and the polymerized ethyl cellulose may further include a long chain fatty acid selected from the group consisting of saturated, unsaturated, and polyunsaturated fatty acids with carbon chain lengths of C₆-C₂₄. Encapsulation of other salts of nicotinamide riboside is contemplated for this embodiment.

In yet another embodiment, the composition comprises polymerized zein protein encapsulating nicotinamide riboside chloride salt. Encapsulation of other salts of nicotinamide riboside is contemplated for this embodiment.

In yet another embodiment, a method is described for making a composition comprising polymerized ethyl cellulose encapsulating nicotinamide riboside chloride salt (NR—Cl).

The steps of the method include: (a) dispersing NR—Cl in a matrix of ethyl cellulose and a solvent to produce a suspension, (b) homogenizing the suspension, (c) atomizing the homogenized suspension by using a spinning disk or spray drying to produce particles of polymerized ethyl cellulose encapsulating NR—Cl, and (d) drying the particles. Optionally, steps (a) through (c) may be repeated to create an outer coating over the dried particles.

In a further embodiment, step (a) may be performed without a solvent where NR—Cl is dispersed in a molten matrix of a biopolymer or other pharmaceutically acceptable polymeric excipient.

In yet another embodiment, a method is described for making a composition comprising polymerized ethyl cellulose encapsulating nicotinamide riboside, a salt thereof, or a solvate thereof, or derivatives thereof (including reduced, 1-4-dihydropyridine forms) comprising the steps of: (a) combining nicotinamide riboside, a salt thereof, or a solvate thereof, with or without ethyl cellulose, and at least one carboxylic acid selected from the group consisting of stearic acid, palmitic acid, myristic acid, lauric acid, capric acid, caprylic acid, caproic acid, oxalic acid, malonic acid, succinic acid, glutaric acid, adipic acid, pimelic acid, suberic acid, fumaric acid, maleic acid, malic acid, tartaric acid, oxaloacetic acid, aspartic acid, glutamic acid, and citric acid; (b) granulating the combined mixture, optionally with a solvent (GRAS acceptable) selected from acetone, methanol, ethanol, or isopropanol (IPA), and the like, to produce particles smaller than 50-150 μm; (c) applying a coating of ethyl cellulose, optionally with an edible oil such as castor oil, to the particles; and (d) drying the particles to provide ethyl cellulose coated particles of nicotinamide riboside, a salt thereof, or a solvate thereof, having an average particle size of about 50-150 μm. In other useful embodiments, the average particle size can range up to about 1000 μm.

In a further embodiment, the method further comprises step (c1) applying a coating of bees wax to the particles, following step (c).

### BRIEF DESCRIPTION OF THE FIGURES

FIG. 1 depicts the NAD⁺ biosynthetic pathway. Nicotinic acid riboside (NAR) and nicotinamide riboside (NR) are shown.

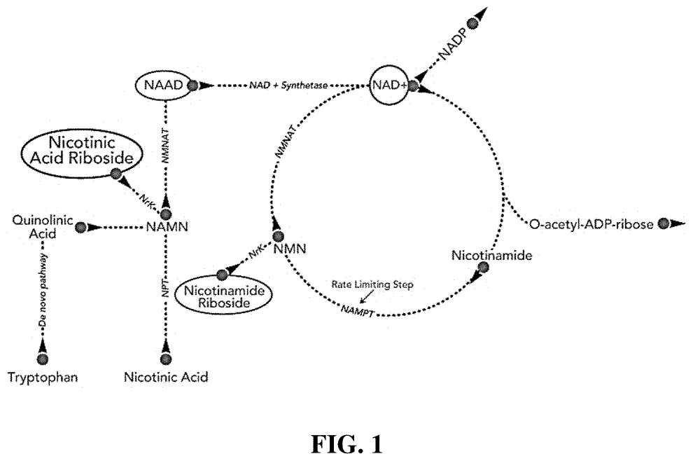

FIG. 2A depicts in one embodiment a SEM photomicrograph (100× magnification) of NR—Cl encapsulated in a 10% NR—Cl, 90% Ethyl Cellulose formulation having a particle size of approx. 50-150 μm.

FIG. 2B depicts in one embodiment a SEM photomicrograph (1000× magnification) of NR—Cl encapsulated in a 10% NR—Cl, 90% Ethyl Cellulose formulation having a particle size of approx. 50-150 μm.

FIG. 3 depicts in one embodiment a total percent mass balance released from 10% NR—Cl in Ethyl Cellulose formulation during a 1-month time course study in water.

FIG. 4A depicts in another embodiment a SEM photomicrograph (100× magnification) of NR—Cl encapsulated in a 10% NR—Cl in Stearic Acid/Ethyl Cellulose formulation having a particle size of approx. 50-200 μm.

FIG. 4B depicts in another embodiment a SEM photomicrograph (1000× magnification) of NR—Cl encapsulated in a 10% NR—Cl in Stearic Acid/Ethyl Cellulose formulation having a particle size of approx. 50-200 μm.

FIG. 5 depicts in another embodiment a total percent mass balance released from 10% NR—Cl in Stearic Acid/Ethyl Cellulose formulation during a 2-week time course study in water.

FIG. 6A depicts in yet another embodiment a SEM photomicrograph (100× magnification) of NR—Cl encapsulated in a 10% NR—Cl/Palmitic Acid/Ethyl Cellulose formulation having a particle size of approx. 50-250 μm.

FIG. 6B depicts in yet another embodiment a SEM photomicrograph (500× magnification) of NR—Cl encapsulated in a 10% NR—Cl/Palmitic Acid/Ethyl Cellulose formulation having a particle size of approx. 50-250 μm.

FIG. 7 depicts in yet another embodiment a total percent mass balance released from 10% NR—Cl in Palmitic Acid/Ethyl Cellulose formulation during a 2-week time course study in water.

FIG. 8A depicts in a further embodiment a SEM photomicrograph (100× magnification) of NR—Cl encapsulated in a 10% NR—Cl in Zein Protein formulation having a particle size of approx. 50-150 μm.

FIG. 8B depicts in a further embodiment a SEM photomicrograph (1000× magnification) of NR—Cl encapsulated in a 10% NR—Cl in Zein Protein formulation having a particle size of approx. 50-150 μm.

FIG. 9 depicts in a further embodiment a total percent mass balance released from 10% NR—Cl in Zein Protein formulation during a 2-week time course study in water.

FIG. 10 depicts in a further embodiment a light microscopy photo (4× magnification) of NR—Cl granulated with ethyl cellulose, 4% fumaric acid and 1% stearic acid and coated with ethyl cellulose and castor oil mixture (about 50% gained by weight) having a particle size of approx. 50-150 μm.

FIG. 11 depicts in a further embodiment a total NR—Cl percent mass balance released from 60.6% NR—Cl formulation in buffered aqueous solution at pH 3.0 over 4 hours.

FIG. 12 depicts in a further embodiment a light microscopy photo (4× magnification) of NR—Cl granulated with ethyl cellulose, 4% fumaric acid and 1% stearic acid and coated with ethyl cellulose and castor oil mixture (about 75% gained by weight) and then with beeswax (about 10% gained by weight) having a particle size of approx. 50-100 μm.

FIG. 13 depicts a further embodiment a total NR—Cl percent mass balance released from 47.2% NR—Cl formulation in buffered aqueous solution at pH 3.0 over 28 days.

FIG. 14 depicts in a further embodiment a light microscopy photo (4× magnification) of NR—Cl granulated with ethyl cellulose, 4% fumaric acid and 1% stearic acid and coated with ethyl cellulose and castor oil mixture (about 100% gained by weight) and then with beeswax (about 10% gained by weight) having a particle size of approx. 50-100 μm.

FIG. 15 depicts a further embodiment a total NR—Cl percent mass balance released from 41.3% NR—Cl formulation in buffered aqueous solution at pH 3.0 over 4 hours.

### DETAILED DESCRIPTION

Nicotinamide riboside (“NR”) is a pyridinium compound having the formula (I):

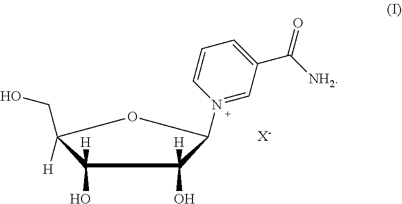

NR of formula (I) can include salts or solvates. Salts may include counterions (defined as “X—”) selected from chloride, bromide, iodide, and the like. For example, one useful salt is the chloride salt of NR (“NR—Cl”). Further salts may include, but are not limited to, fluoride, formate, acetate, propionate, butyrate, glutamate, aspartate, ascorbate, benzoate, carbonate, citrate, carbamate, gluconate, lactate, methyl bromide, methyl sulfate, nitrate, phosphate, diphosphate, succinate, sulfate, tartrate, hydrogen tartrate, malate, hydrogen malate, maleate, fumarate, citrate, stearate, palmitate, myristate, laurate, caprate, caprylate, caproate, oleate, linoleate, sulfonate, trifluoromethanesulfonate, trichloromethanesulfonate, tribromomethanesulfonate, trichloroacetate, tribromoacetate, and trifluoroacetate. For NAR, NAMN and NMN, and the like, optionally wherein when X— is absent, optionally the counterion is an internal salt, and/or zwitterion.

NR is hydrophilic, although susceptible to hydrolysis. This presents a unique requirement such that chemical stability requires microencapsulation of a water soluble compound. This is a reversal of the common formulator's technique to microencapsulate a hydrophobic or water-insoluble material in order to provide better bioavailability.

In a further aspect, derivatives of NR are contemplated having the formula (Ia) or a salt, solvate, or prodrug thereof:

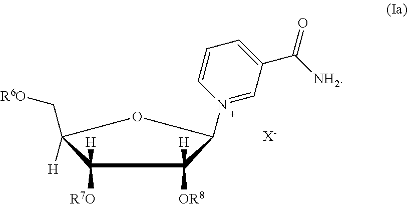

wherein R⁶ is selected from the group consisting of hydrogen, —C(O)R′, —C(O)OR′, —C(O)NHR′, substituted or unsubstituted (C₁-C₅)alkyl, substituted or unsubstituted (C₁-C₅)cycloalkyl, substituted or unsubstituted aryl, substituted or unsubstituted heteroaryl, and substituted or unsubstituted heterocycle;

R′ is selected from the group consisting of hydrogen, —(C₁-C₈)alkyl, —(C₁-C₈)cycloalkyl, aryl, heteroaryl, heterocycle, aryl(C₁-C₄)alkyl, and heterocycle(C₁-C₄)alkyl; and

R₇ and R₈ are independently selected from the group consisting of hydrogen, —C(O)R′, —C(O)OR′, —C(O)NHR′, substituted or unsubstituted (C₁-C₂₄)alkyl, substituted or unsubstituted (C₁-C₈)cycloalkyl, substituted or unsubstituted aryl, substituted or unsubstituted heteroaryl, substituted or unsubstituted heterocycle, substituted or unsubstituted aryl(C₁-C₄)alkyl, and substituted or unsubstituted heterocycle(C₁-C₄)alkyl. Salts may include counterions (defined as “X”) selected from chloride, bromide, iodide, and the like, as discussed above.

This disclosure also includes other NAD+ precursors, such as, but not limited to, one or more nicotinyl riboside compounds selected from nicotinic acid riboside (NAR, II), nicotinamide mononucleotide (NMN, III), nicotinic acid mononucleotide (NaMN, IV), reduced nicotinamide riboside (NRH, V), reduced nicotinic acid riboside (NARH, VI), NR triacetate (NRTA, VII which is a species of Ia), NAR triacetate (NARTA, VIII), NRH triacetate (NRH-TA, IX), or NARH triacetate (NARH-TA, X), and salts, solvates, or mixtures thereof, or derivatives thereof.

Nicotinic acid riboside (NAR) is a pyridinium nicotinyl compound having the formula (II):

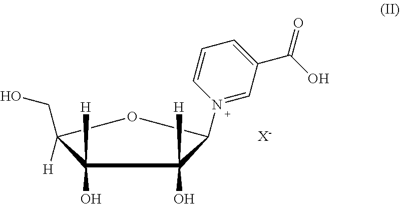

Nicotinamide mononucleotide (NMN) is a pyridinium nicotinyl compound having the formula (III):

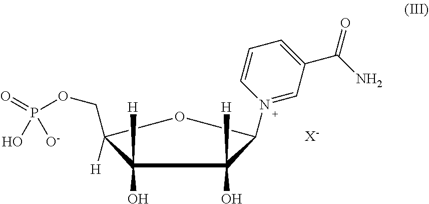

Nicotinic acid mononucleotide (NaMN) is a pyridinium nicotinyl compound having the formula (IV):

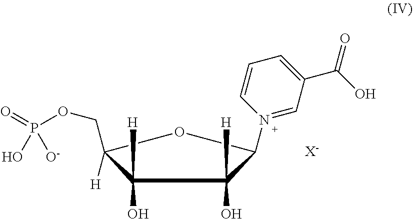

Reduced nicotinamide riboside (“NRH”) is a 1,4-dihydropyridyl reduced nicotinyl compound having the formula (V):

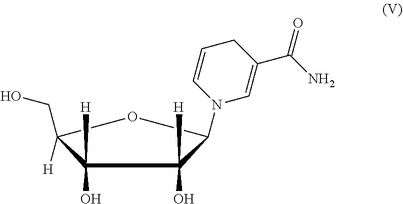 [SMILES: NC(=O)C1=CN([C@@H]2O[C@H](CO)[C@@H](O)[C@@H]2O)C=CC1]

Reduced nicotinic acid riboside (“NARH”) is a 1,4-dihydropyridyl reduced nicotinyl compound having the formula (VI):

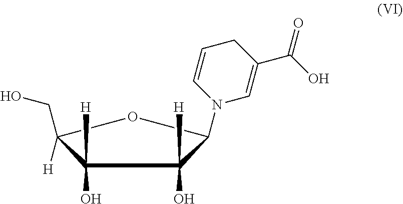 [SMILES: O=C(O)C1=CN([C@@H]2O[C@H](CO)[C@@H](O)[C@@H]2O)C=CC1]

In a species of compound (Ia), the free hydrogens of hydroxyl groups on the ribose moiety of nicotinamide riboside (NR, I) can be substituted with acetyl groups (CH₃—C(═O)—) to form 1-(2′,3′,5′-triacetyl-beta-D-ribofuranosyl)-nicotinamide (“NR triacetate” or “NRTA”) having the formula (VII):

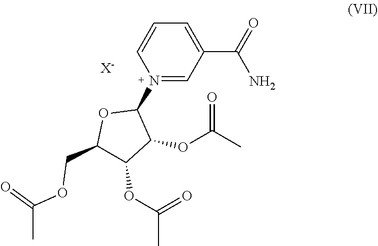

The free hydrogens of hydroxyl groups on the ribose moiety of nicotinic acid riboside (NAR, II) can be substituted with acetyl groups (CH₃—C(═O)—) to form 1-(2′,3′,5′-triacetyl-beta-D-ribofuranosyl)-nicotinic acid (“NAR triacetate” or “NARTA”) having the formula (VIII):

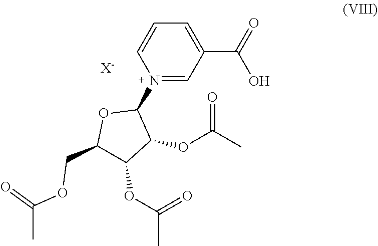

The free hydrogens of hydroxyl groups on the ribose moiety of reduced nicotinamide riboside (NRH, V) can be substituted with acetyl groups (CH₃—C(═O)—) to form 1-(2′,3′,5′-triacetyl-beta-D-ribofuranosyl)-1,4-dihydronicotinamide (“NRH triacetate” or “NRH-TA”) having the formula (IX):

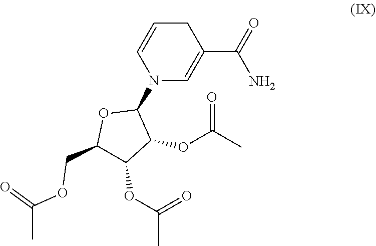 [SMILES: CC(=O)OC[C@H]1O[C@@H](N2C=CCC(C(N)=O)=C2)[C@H](OC(C)=O)[C@@H]1OC(C)=O]

The free hydrogens of hydroxyl groups on the ribose moiety of reduced nicotinic acid riboside (NARH, VI) can be substituted with acetyl groups (CH₃—C(═O)—) to form 1-(2′,3′,5′-triacetyl-beta-D-ribofuranosyl)-1,4-dihydronicotinic acid (“NARH triacetate” or “NARH-TA”) having the formula (X):

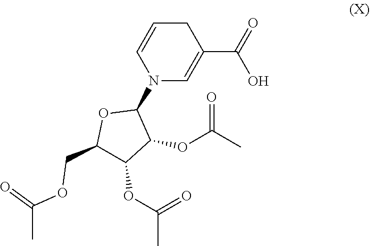 [SMILES: CC(=O)OC[C@H]1O[C@@H](N2C=CCC(C(=O)O)=C2)[C@H](OC(C)=O)[C@@H]1OC(C)=O]

For each of nicotinamide riboside (NR, I), nicotinic acid riboside (NAR, II), nicotinamide mononucleotide (NMN, III), nicotinic acid mononucleotide (NaMN, IV), reduced nicotinamide riboside (NRH, V), reduced nicotinic acid riboside (NARH, VI), nicotinamide riboside triacetate (NRTA, VII), nicotinic acid riboside triacetate (NARTA, VIII), reduced nicotinamide riboside triacetate (NRH-TA, IX), and reduced nicotinic acid riboside triacetate (NARH-TA, X), optionally X— as counterion is absent, or when X— is present, X— is selected from the group consisting of bromide, iodide, fluoride, formate, acetate, propionate, butyrate, glutamate, aspartate, ascorbate, benzoate, carbonate, citrate, carbamate, gluconate, lactate, methyl bromide, methyl sulfate, nitrate, phosphate, diphosphate, succinate, sulfate, tartrate, hydrogen tartrate, malate, hydrogen malate, maleate, fumarate, citrate, stearate, palmitate, myristate, laurate, caprate, caprylate, caproate, oleate, linoleate, sulfonate, trifluoromethanesulfonate, trichloromethanesulfonate, tribromomethanesulfonate, trichloroacetate, tribromoacetate, and trifluoroacetate; and,

optionally wherein when X— is absent, optionally the counterion is an internal salt or zwitterion;

optionally X⁻ is an anion of a substituted or unsubstituted carboxylic acid selected from monocarboxylic acid, a dicarboxylic acid, or a polycarboxylic acid;

optionally X⁻ is an anion of a substituted monocarboxylic acid, further optionally an anion of a substituted propanoic acid (propanoate or propionate), or an anion of a substituted acetic acid (acetate), or an anion of a hydroxyl-propanoic acid, or an anion of 2-hydroxypropanoic acid (being lactic acid; the anion of lactic acid being lactate), or a trihaloacetate selected from trichloroacetate, tribromoacetate, or trifluoroacetate; and,

optionally X⁻ is an anion of an unsubstituted monocarboxylic acid selected from formic acid, acetic acid, propionic acid, or butyric acid, the anions being formate, acetate, propionate, butyrate, and stearate, and the like, respectively; or an anion of a long chain fatty acid including saturated, unsaturated and polyunsaturated fatty acids with carbon chain lengths of C₆-C₂₄ (such as, for example, stearic acid, palmitic acid, myristic acid, lauric acid, capric acid, caprylic acid, caproic acid, oleic acid, linoleic acid, omega-6 fatty acid, omega-3 fatty acid; the anions being stearate, palmitate, myristate, laurate, caprate, caprylate, caproate, oleate, linoleate, etc.); and,

optionally X— is an anion of a substituted or unsubstituted amino acid, i.e., amino-monocarboxylic acid or an amino-dicarboxylic acid, optionally selected from glutamic acid and aspartic acid, the anions being glutamate and aspartate, respectively; or, alternatively, selected from alanine, beta-alanine, arginine, asparagine, cysteine, glutamine, glycine, histidine, isoleucine, leucine, lysine, methionine, phenylalanine, proline, serine, threonine, tryptophan, or tyrosine, and,

optionally X⁻ is an anion of ascorbic acid, being ascorbate; and,

optionally X⁻ is a halide selected from fluoride, chloride, bromide, or iodide; and,

optionally X⁻ is an anion of a substituted or unsubstituted sulfonate, further optionally a trihalomethanesulfonate selected from trifluoromethanesulfonate, tribromomethanesulfonate, or trichloromethanesulfonate; and,

optionally X⁻ is an anion of a substituted or unsubstituted carbonate, further optionally hydrogen carbonate.

For each of the forgoing structures (II), (III), (IV), (V), (VI), (VII), (VIII), (IX), and (X), substituted derivatives and/or analogues thereof, respectively, are contemplated as useful for encapsulated compositions, as described herein, or useful for methods of making polymerized materials encapsulating said compounds, as described herein.

Microencapsulation

Microencapsulation is a technique used for the protection of a wide range of biomolecules.

Formulations may be prepared as any product form suitable for use in human individuals, including reconstitutable powders, ready-to-drink liquids, parenteral (intravenous) formulations, and dilutable liquid concentrates, product forms which are all well known in the nutritional formula art. As used in the present application, the amounts of components present in formulations or compositions refer to the amounts when the formulation or composition is ready for consumption by the human individual.

In various embodiments, a nutraceutical comprising one or more nicotinyl riboside compounds (I, Ia, II, III, IV, V, VI, VII, VIII, IX, and/or X), or salts thereof, alone or in combination with one or more vitamins), may be any variety of food or drink. For example, nutraceuticals may include drinks such as nutritional drinks, diet drinks (e.g., Slimfast™, Boost™, and the like) as well as sports (e.g., Gatorade™, Powerade™, EAS™, and the like), herbal, medical (e.g., Ensure™, Optifast™), and other fortified beverages (e.g., MuscleMilk™ Pedialyte™). Additionally, nutraceuticals may include food intended for human or animal consumption such as baked goods, for example, bread, wafers, cookies, crackers, pretzels, pizza, and rolls; ready-to-eat (“RTE”) breakfast cereals, hot cereals; pasta products; snacks such as fruit snacks, salty snacks, grain snacks, nutrition bars, and microwave popcorn; dairy products such as yogurt, cheese, and ice cream; sweet goods such as hard candy, soft candy, and chocolate; beverages; animal feed; pet foods such as dog food and cat food; aqua-culture foods such as fish food and shrimp feed; and special purpose foods such as baby food (e.g., Gerber™), infant formulas (e.g., Good Start™, Similac™, Enfamil™), hospital food, medical food, sports food, performance food, or nutritional bars; fortified foods; food preblends; or mixes for home or food service use, such as preblends for soups or gravy, dessert mixes, dinner mixes, baking mixes such as bread mixes and cake mixes, and baking flour. In certain embodiments, the food or beverage does not include one or more of grapes, mulberries, blueberries, raspberries, peanuts, yeast, or extracts thereof.

Useful vitamins may include Vitamin B3, which is also known as “nicotinic acid,” or “niacin,” and is a pyridine compound. It will be apparent to those skilled in the art that vitamin B3 is functionally and chemically inequivalent to, and not interchangeable with, nicotinamide riboside (NR, I), NR—X salts, or derivatives thereof. Other useful vitamins include Vitamins B1, B2, B6, B7, B9, B12, A₁, C, D₃, D₂, E, and K₁.

Without being bound by theory, vitamins B1, B2, B3, and B6 are believed to be closely intertwined in their biosynthetic pathways, with the maintenance and regeneration of the NAD(P)(H) intracellular pool depending on the availability of ThDP (B1), FAD (B2), and PLP (B6). Thiamine (vitamin B1), riboflavin (vitamin B2), and pyridoxine (vitamin B6) are salvaged from food and converted back intracellularly to their respective, bioactive forms: Thiamine (ThDP); Flavin Adenine Dinucleotide (FAD); Nicotinamide Adenine Dinucleotide (NAD⁺); and PyridoxaL Phosphate (PLP). The conversion of vitamins B1, B2, and B6 to ThDP, FAD, and PLP, respectively, is ATP-dependent. Two of the three salvage pathways that convert vitamin B3 to NAD⁺ are dependent on ThDP (B1), with the de novo production of NAD⁺ from tryptophan depending on the bioactive forms of vitamins B1, B2, and B6. The vitamin B1 dependency comes from the fact that ThDP (B1) is cofactor for the transketolases involved in the biosynthesis of phosphoriboside pyrophosphate, an essential substrate in these aforementioned NAD⁺ salvage and de novo pathways.

Certain useful biopolymers, or semi-synthetic biopolymers, used for microencapsulation herein include, but are not limited to, ethyl cellulose, ethyl cellulose combined or doped with a long chain fatty acid including saturated, unsaturated and polyunsaturated fatty acids with carbon chain lengths of C₆-C₂₄ (such as, for example, stearic acid, palmitic acid, myristic acid, lauric acid, capric acid, caprylic acid, caproic acid), or dicarboxylic acids, such as oxalic acid, malonic acid, succinic acid, glutaric acid, adipic acid, pimelic acid, suberic acid, fumaric acid, maleic acid, malic acid, tartaric acid, oxaloacetic acid, aspartic acid, glutamic acid, or tricarboxylic acids, such as citric acid, Eudragaurd® (methacrylate copolymer, Evonik Industries), Eudragit®, chitosan, and zein protein. In an embodiment, high density cross-linked polymers are desirable components. Other useful pharmaceutically acceptable polymeric excipients are contemplated for use with the present invention.

One type of useful ethyl cellulose polymer is ETHOCEL™ (a.k.a. as “Ethocel”, available from DuPont, Wilmington, Del.). ETHOCEL™ ethylcellulose polymers are organosoluble products with a narrow specification and uniform ethoxyl functionality. This enables ETHOCEL™ to have very predictable performance, key to industrial applications where the same output is desired batch to batch. ETHOCEL™ polymers are water-insoluble thermoplastic polymers; so they can therefore be employed for a wide variety of functions. They are thermally stable and form scuff-resistant, flexible coatings at low temperatures. They are used for rheology modification, film formation, binding, water barriers, and as time-release agents. ETHOCEL™ can also be effectively used as a sacrificial binder as they exhibit clean burn out.

In one embodiment, the amount of NR (such as NR—Cl, or other nicotinyl riboside derivative), or a salt, solvate, or prodrug thereof, can be present in an amount of about 0.1% by weight to about 90% by weight of the encapsulated material in combination with one or more biopolymers. In a preferred embodiment, the amount of NR (such as NR—Cl, or other nicotinyl riboside derivative), or a salt, solvate, or prodrug thereof, can be present in an amount of about 0.1% by weight to about 65% by weight of the encapsulated material in combination with one or more biopolymers. In certain embodiments, the amount of NR (such as NR—Cl, or other nicotinyl riboside derivative), or a salt, solvate, or prodrug thereof, can be present in an amount of about 30% by weight, or 35% by weight, or 40% by weight, or 45 by weight, or 50% by weight, or 55% by weight, or 60% by weight, or 65% by weight, or 70% by weight, or 75% by weight, or 80% by weight, or 85% by weight of the encapsulated material in combination with one or more biopolymers or semi-synthetic polymers.

Useful ratios of ethyl cellulose (Ethocel) with fatty acids may include 1:99 Ethocel:Fatty Acid (wt/wt) up to about 100% Ethocel.

In an embodiment, edible oils are contemplated to be used in combination with ethyl cellulose (Ethocel), in similar ratios of 1:99 Ethocel:oil (wt/wt) up to about 100% Ethocel. Useful edible oils may include, but are not limited to, castor oil, palm oil, sunflower oil, carnauba wax, cottonseed oil, soybean oil, cocoa butter, paraffin wax, bees wax, high oleic safflower oil, soy oil, fractionated coconut oil, medium chain triglycerides, MCT oil, high oleic sunflower oil, corn oil, canola oil, coconut oil, palm kernel oil, marine oil, walnut oil, wheat germ oil, sesame oil, cod liver oil, candelilla wax, palm stearin, rapeseed oil, glycerol dibehenate, glycerol distearate, peanut oil, and the like, or mixtures of such oils. Additional useful edible oils include food oil, vegetable oil, botanical oil, grape seed oil, sesame oil, borage oil, fish oil, sea buckthorne oil, flax oil, peanut oil, jojoba oil, corn oil, evening primrose oil, amaranth oil, safflower oil, soybean oil, palm oil, almond oil, cashew oil, hazelnut oil, macademia oil, pecan oil, pistachio oil, acai oil, blackcurrant oil, apricot oil, argan oil, artichoke oil, avocado oil, babassu oil, ben oil, borneo tallow nut oil, buffalo gourd oil, carob pod oil, coriander seed oil, false flax oil, hemp oil, kapok seed oil, lallemantia oil, meadowfoam seed oil, mustard oil, okra seed oil, perilla seed oil, pequi oil, pine nut oil, poppy seed, prune kernel oil, pumpkin seed oil, quinoa oil, ramtil oil, rice bran oil, tea oil, thistle oil, and the like, or mixtures of such oils.

Formulations or compositions can optionally be sterilized and subsequently used on a ready-to-feed basis, or can be stored as concentrates. Concentrates can be prepared by spray drying a liquid formulation prepared as above, and a formulation can be reconstituted by rehydrating the concentrate. The formulation concentrate is a stable liquid and has a suitable shelf life.

Compositions for oral formulations useful for delivering an NR-containing composition can be orally administered, for example, with an inert diluent or with an assimilable edible carrier, or it can be enclosed in hard or soft shell gelatin or hydroxypropyl methylcellose (i.e., hypromellose) capsules, or it can be compressed into tablets, or it can be incorporated directly with the food of the diet. For oral administration, an NR-containing composition may be incorporated with an excipient and used in the form of ingestible tablets, buccal tablets, troches, capsules, elixirs, suspensions, syrups, wafers, and the like. The tablets, troches, pills, capsules, and the like can also contain the following: a binder such as gum tragacanth, acacia, corn starch, or gelatin; excipients such as dicalcium phosphate, microcrystalline cellulose, and the like; a disintegrating agent such as potato starch, alginic acid, and the like; a lubricant such as magnesium stearate; and a sweetening agent such as sucrose, lactose, or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the active compound, sucrose as a sweetening agent, methyl and propylparabens as preservatives, a dye, and flavoring such as cherry or orange flavor. Oil-in-water emulsions may be better for oral use in infants because these are water-miscible, and thus their oiliness is masked. Such emulsions are well known in the pharmaceutical sciences.

One goal of the present process for encapsulation of NR is to prevent, limit, or minimize leaching of the product while in storage or in use. Another goal of the present process is to enhance stability and shelf-life.

The compositions and methods described in the embodiments above may be further understood in connection with the following Examples. In addition, the following non-limiting examples are provided to illustrate the invention. However, the person skilled in the art will appreciate that it may be necessary to vary the procedures for any given embodiment of the invention, e.g., vary the order or steps of the methods.

### Example 1

Ethyl Cellulose

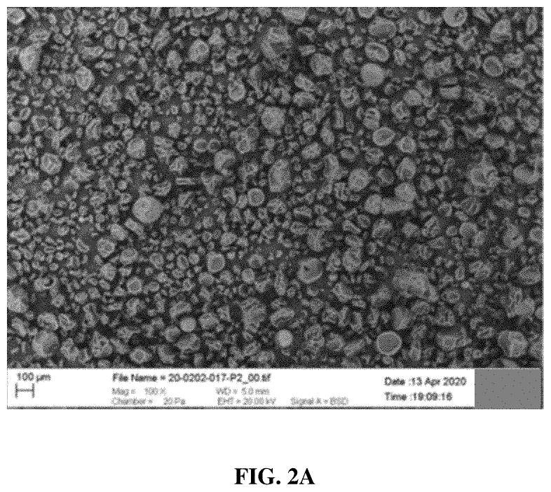

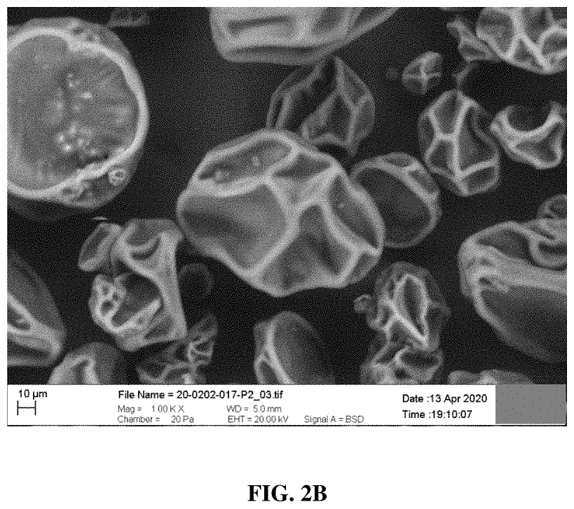

Jet milled NR—Cl with a particle size of approx. 5-10 μm was encapsulated in a 10% NR—Cl, 90% Ethyl Cellulose formulation. Ethyl Cellulose (Ethocel, available from DuPont, Wilmington, Del.) was first dissolved into ethanol at 10% wt/wt. Once fully dissolved, NR—Cl was dispersed into the ethanol solution and the suspension was homogenized and fed into a spinning disk for atomization. The resulting dried formulation contained 1.04% (wt/wt) water, and produced particles ranging from 1 to 200 μm, with a majority of the particles from 50 to 150 μm in diameter (FIGS. 2A, 2B).

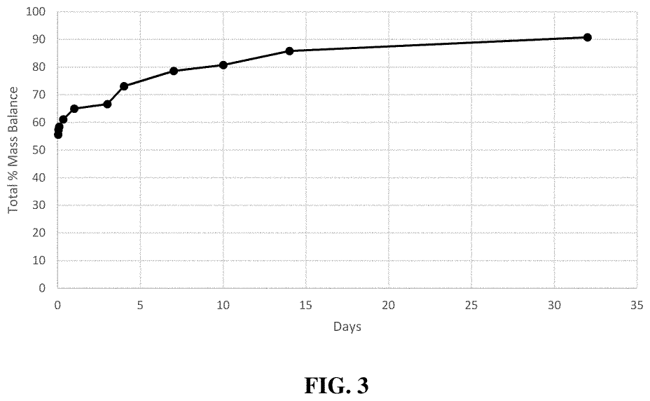

To test the retention of NR—Cl in the formulation, the formulation was added to water at ambient temperature. The water solution was analyzed for NR—Cl and its constituents to calculate mass balance recovery during a time course study (Table 1 and FIG. 3). The Ethyl Cellulose formulation only released approximately 60% of the NR—Cl in the formulation after 8 hours. Over the course of 32 days, additional NR—Cl was gradually released. At 32 days, approximately 90% of the NR—Cl had been released.

TABLE 1

| | % Recovery |
|---|---|
| | (Mass |
| Days | Balance) |
| 0.02 | 55.6 |
| 0.04 | 57.4 |
| 0.08 | 58.4 |
| 0.33 | 61.1 |
| 1 | 65.0 |
| 3 | 66.6 |
| 4 | 73.1 |
| 7 | 78.6 |
| 10 | 80.7 |
| 14 | 85.8 |
| 32 | 90.8 |

Table 1 shows total mass balance percent recovered from the 10% NR—Cl in Ethyl Cellulose Formulation in a water time course study.

### Example 2

Ethyl Cellulose/Stearic Acid

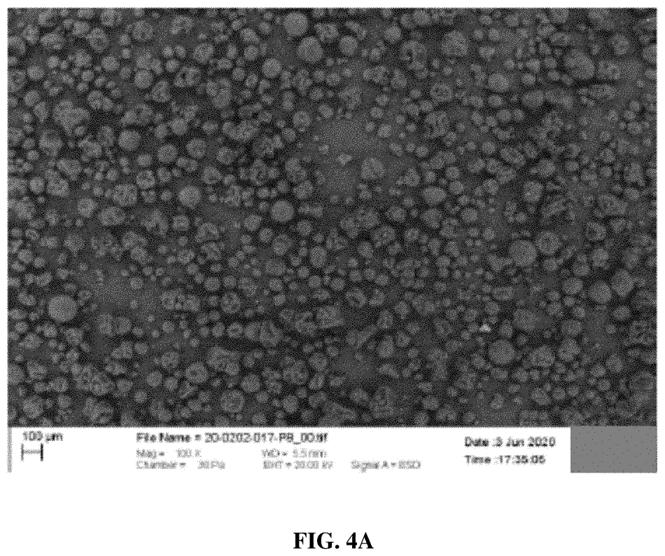

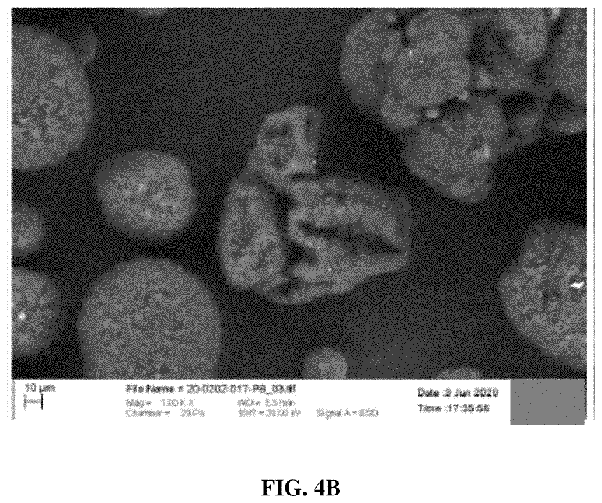

Jet milled NR—Cl with a particle size of approx. 5-10 μm was encapsulated in a 10% NR—Cl, 90% Stearic Acid/Ethyl Cellulose formulation. NR—Cl was first dispersed into molten Stearic Acid at 15% wt/wt. Once fully homogenized, the suspension was fed into a heated spinning disk. The resulting material was dispersed into ethanol containing 8% ethyl cellulose wt/wt, and again fed into a spinning disk to create a final formulation containing 10% NR—Cl. The resulting dried formulation contained 0.60% (wt/wt) water, and produced particles ranging from 10 to 300 μm, with a majority of the particles from 50 to 200 μm in diameter (FIGS. 4A, 4B).

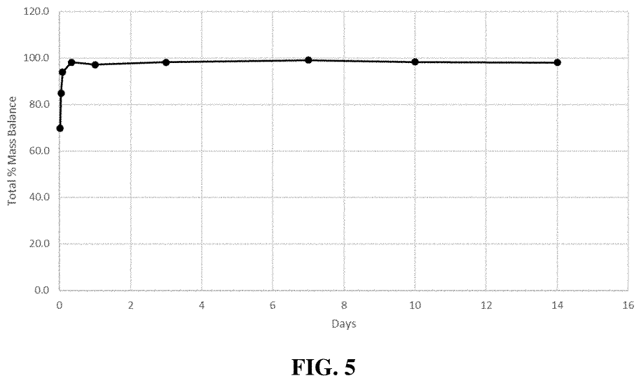

To test the retention of NR—Cl in the Stearic Acid/Ethyl Cellulose formulation, the formulation was added to water at room temperature. The water solution was analyzed for NR—Cl and its constituents to calculate mass balance recovery during a time course study (Table 2 and FIG. 5). The Steric Acid/Ethyl Cellulose formulation released approximately 98% of the NR—Cl after 8 hours, and maintained mass balance through 14 days.

TABLE 2

| | % Recovery |
|---|---|
| | (Mass |
| Days | Balance) |
| 0.02 | 69.7 |
| 0.04 | 84.7 |
| 0.08 | 93.8 |
| 0.33 | 98.1 |
| 1 | 97.1 |
| 3 | 98.1 |
| 7 | 99.1 |
| 10 | 98.3 |
| 14 | 98.0 |

Table 2 shows total mass balance percent recovered from the 10% NR—Cl in Stearic Acid/Ethyl Cellulose Formulation in a water time course study.

### Example 3

Ethyl Cellulose/Palmitic Acid

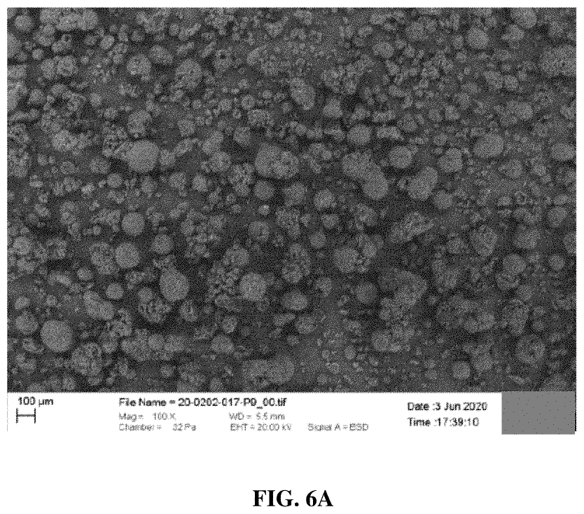

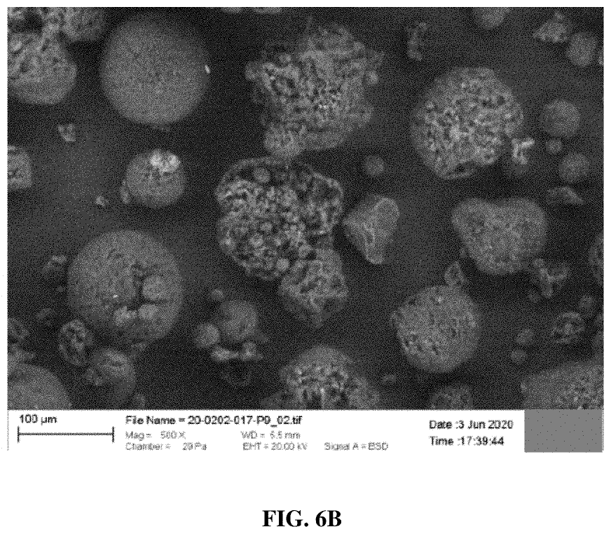

Jet milled NR—Cl with a particle size of approx. 5-10 μm was encapsulated in a 10% NR—Cl, 90% Palmitic Acid/Ethyl Cellulose formulation. First, NR—Cl was dispersed into molten palmitic acid at 15% (wt/wt). Once fully homogenized, the suspension was fed into a heated spinning disk. The resulting material was dispersed into ethanol containing 8% ethyl cellulose wt/wt, and again fed into a spinning disk to create a final formulation containing 10% NR—Cl. The resulting dried formulation contained 0.72% (wt/wt) water, and produced particles ranging from 10 to 400 μm, with a majority of the particles from 50 to 250 μm in diameter (FIGS. 6A, 6B).

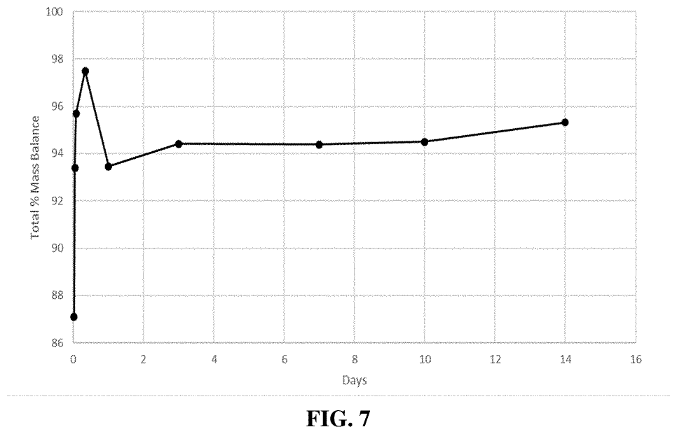

To test the retention of NR—Cl in the formulation, the formulation was added to water at ambient temperature. The water solution was analyzed for NR—Cl and its constituents to calculate mass balance recovery during a time course study (Table 3 and FIG. 7). The Palmitic acid/Ethyl Cellulose formulation released approximately 97% of the NR—Cl in the formulation after 8 hours, and maintained mass balance through 14 days.

TABLE 3

| | % Recovery |
|---|---|
| | (Mass |
| Days | Balance) |
| 0.02 | 87.1 |
| 0.04 | 93.4 |
| 0.08 | 95.7 |
| 0.33 | 97.5 |
| 1 | 93.5 |
| 3 | 94.4 |
| 7 | 94.4 |
| 10 | 94.5 |
| 14 | 95.3 |

Table 3 shows total mass balance percent recovered from the 10% NR—Cl in Palmitic Acid/Ethyl Cellulose Formulation in a water time course study.

### Example 4

Zein Protein

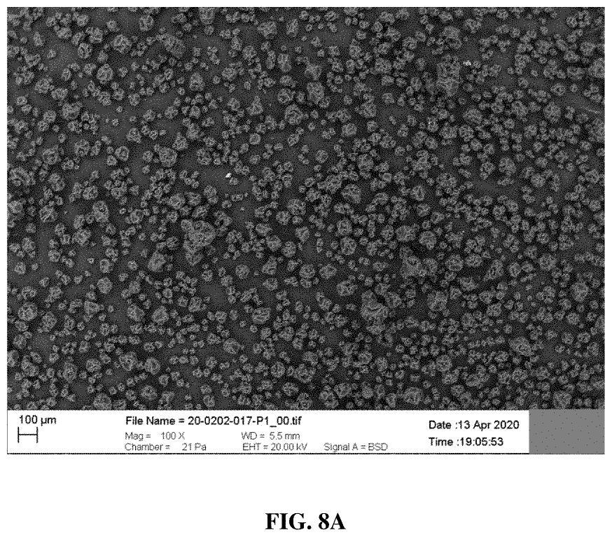

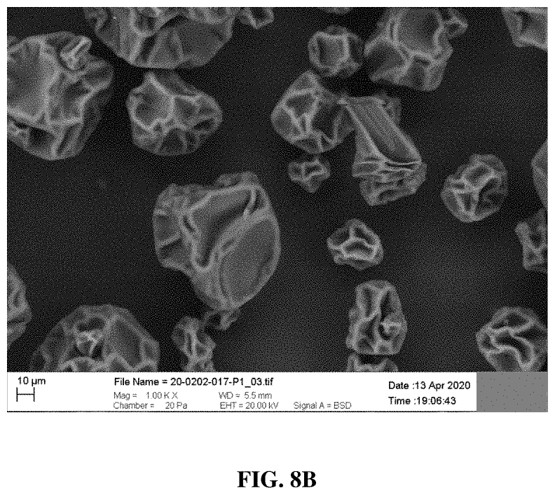

Jet milled NR—Cl with a particle size of approx. 5-10 μm was encapsulated in a 10% NR—Cl, 90% Zein Protein formulation. Zein (available from Flo Chemical Corporation, Ashburnham, Mass.) was first dissolved in 10/90 water/ethanol. Once fully dissolved, NR—Cl was dispersed into the water/ethanol solution and the suspension was homogenized and fed into a spinning disk for atomization. The resulting dried formulation contained 1.68% (wt/wt) water, and produced particles ranging from 1 to 200 μm, with a majority of the particles from 50 to 150 μm in diameter (FIGS. 8A, 8B).

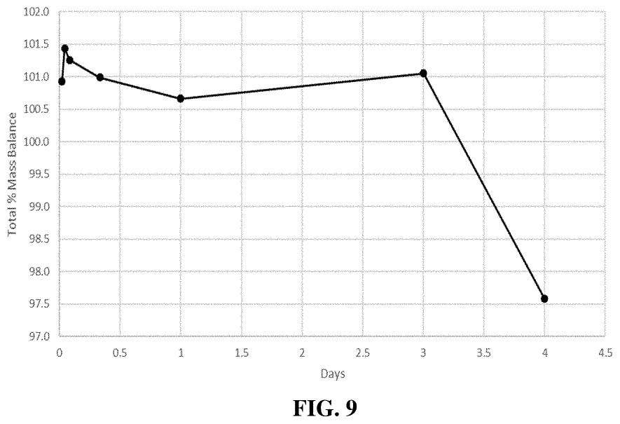

To test the retention of NR—Cl in the formulation, the formulation was added to water at ambient temperature. The water solution was analyzed for NR—Cl and its constituents to calculate mass balance recovery during a time course study (Table 4 and FIG. 9). The Zein formulation released approximately 100% of the NR—Cl in the formulation by the 30 min time point, and maintained mass balance through 4 days.

TABLE 4

| | % Recovery |
|---|---|
| | (Mass |
| Days | Balance) |
| 0.02 | 100.9 |
| 0.04 | 101.4 |
| 0.08 | 101.3 |
| 0.33 | 101.0 |
| 1 | 100.7 |
| 3 | 101.1 |
| 4 | 97.6 |

Table 4 shows total mass balance percent recovered from the 10% NR—Cl in Zein Protein formulation in a water time course study.

### Example 5

Chemical characterization of microencapsulated NR samples, or salts thereof, or solvates thereof, or derivatives thereof. It is well understood that samples made in accordance with the principles of this disclosure may be characterized by various means well known in the art, including, but not limited to, viscosity measurements and other rheological measurements, FT-IR, NMR, HPLC, mass spectrometry, LC/MS techniques, TEM/SEM photomicrography, light microscopy, microstructure and morphology studies, stability studies (solid phase, solution phase, humidity, thermal), particle size, Zeta potential, thermal characterization (thermogravimetric analysis (TGA), differential scanning calorimetry (DSC)), flowability, and the like. It is expected that the said chemical analyses will further show the unique qualities and properties of the compositions described herein. In the embodiments described herein, the average particle size may range from about 50 μm up to about 1000 μm.

### Example 6

Ethyl Cellulose Coating/Fumaric Acid/Stearic Acid/Castor Oil, Providing a 60.6% NR—Cl by Weight Formulation

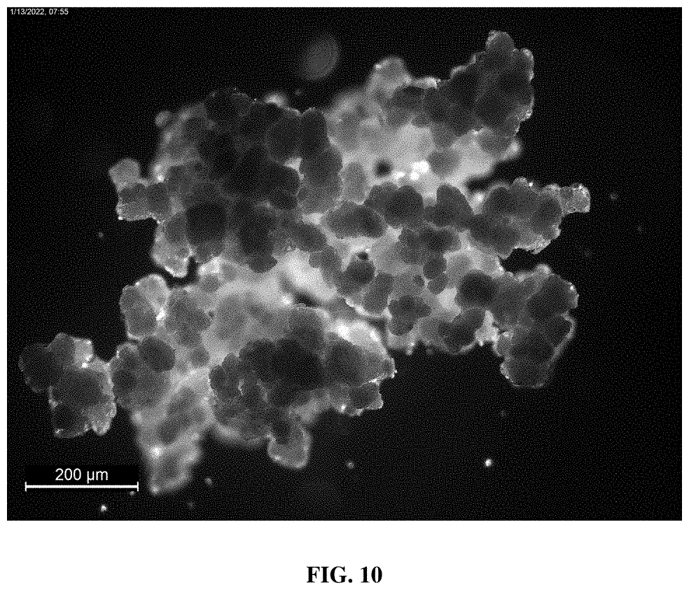

NR—Cl was granulated in Isopropyl alcohol with ethyl cellulose, 4% fumaric acid and 1% stearic acid, resulting in a ˜10% weight gain to the NR—Cl. Then, an 87% Ethyl cellulose and 13% castor oil coating was applied utilizing a fluidized bed technology targeting a 50% weight gain. The final formulation had a theoretical NR—Cl of 60.6% (w/w) and a particle size of approximately 50-100 μm in diameter (FIG. 10).

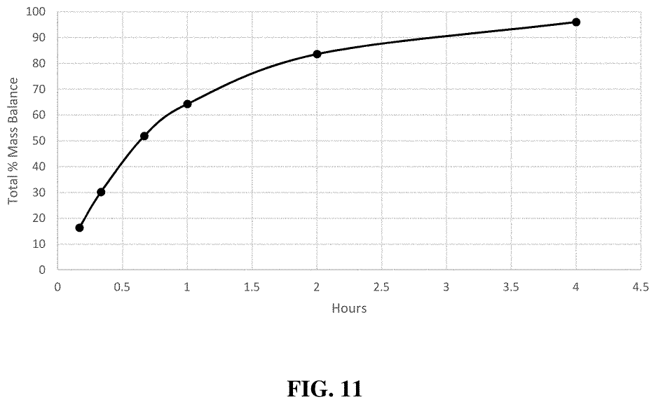

To evaluate the retention of NR—Cl, the formulation was added to an aqueous 10 mM phosphate solution, buffered at pH 3.0. The solution was analyzed for NR—Cl and its constituents to calculate a mass balance recovery over four hours held at ambient conditions with minimal agitation (Table 5 and FIG. 11). The 50% Ethyl Cellulose formulation released approximately 64.3% (w/w) of the initial NR—Cl in the formulation after 1. Over the course of 4 hours, additional NR—Cl was released. At four hours, 96% (w/w) of the NR—Cl had been released.

TABLE 5

| | % Recovery |
|---|---|
| | (Mass |
| Hours | Balance) |
| 0.17 | 16.4 |
| 0.33 | 30.2 |
| 0.67 | 51.8 |
| 1.00 | 64.3 |
| 2.00 | 83.6 |
| 4.00 | 96.0 |

Table 5 shows total NR—Cl percent mass balance recovered from the 60.6% NR—Cl formulation in 10 mM Phosphate buffered aqueous solution at a pH of 3.0 over four hours at ambient conditions.

FIG. 11 shows total NR—Cl percent mass released from the 60.6% NR—Cl formulation in 10 mM phosphate buffered aqueous solution at pH 3.0 over four hours at ambient conditions.

### Example 7

Ethyl Cellulose Coating Plus Beeswax/Fumaric Acid/Stearic Acid/Castor Oil, Providing a 47.2% NR—Cl by Weight Formulation

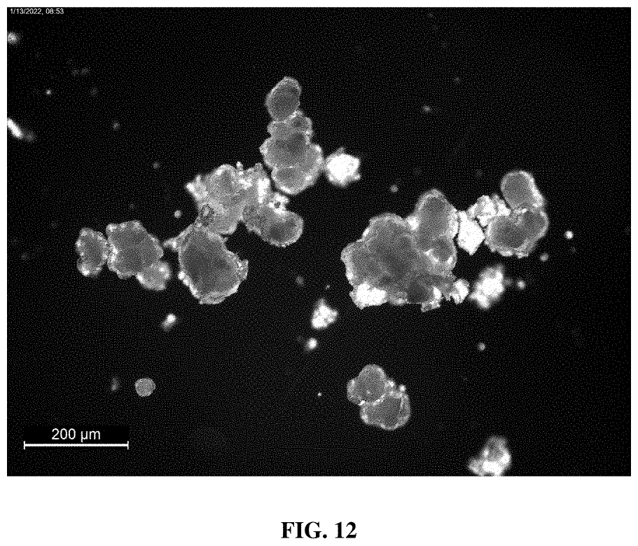

NR—Cl was granulated in Isopropyl alcohol with ethyl cellulose, 4% fumaric acid and 1% stearic acid, resulting in a ˜10% weight gain to the NR—Cl. Then, an 87% Ethyl cellulose and 13% castor oil coating was applied utilizing a fluidized bed technology targeting a 75% weight gain. Then, a Beeswax coating was applied over the ethyl cellulose targeting an additional 10% weight gain. The final formulation had a theoretical NR—Cl of 47.2%. (w/w) and a particle size of approximately 50-100 μm in diameter (FIG. 12).

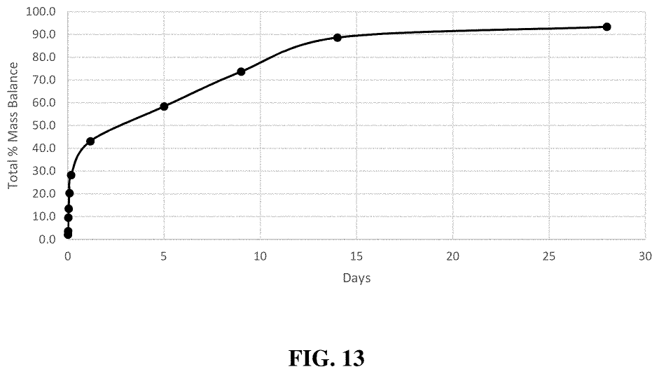

To evaluate the retention of NR—Cl, the formulation was added to an aqueous 10 mM phosphate solution, buffered at pH 3.0. The solution was analyzed for NR—Cl and its constituents to calculate a mass balance recovery over 28 days at ambient conditions with minimal agitation (Table 6 and FIG. 13). The 75% Ethyl Cellulose/10% Wax formulation released approximately 58% (w/w) of the initial NR—Cl in the formulation after 5 days. Over the course of 28 days, additional NR—Cl was gradually released. At 28 days, 93.4% (w/w) of the NR—Cl had been released.

TABLE 6

| | % Recovery |
|---|---|
| | (Mass |
| Days | Balance) |
| 0.007 | 2.1 |
| 0.014 | 3.6 |
| 0.028 | 9.6 |
| 0.042 | 13.5 |
| 0.083 | 20.3 |
| 0.167 | 28.2 |
| 1.167 | 43.1 |
| 5 | 58.4 |
| 9 | 73.6 |
| 14 | 88.6 |
| 28 | 93.4 |

Table 6 shows NR—Cl percent mass balance recovered from the 47.2% NR—Cl formulation in a 10 mM Phosphate buffered aqueous solution at a pH of 3.0 over 28 days at ambient conditions.

FIG. 13 shows total NR—Cl percent mass balance released from the 47.2% NR—Cl formulation in a 10 mM phosphate buffered aqueous solution at a pH 3.0 over 28 days at ambient conditions.

### Example 8

Ethyl Cellulose Coating Plus Beeswax/Fumaric Acid/Stearic Acid/Castor Oil, Providing a 41.3% NR—Cl by Weight Formulation

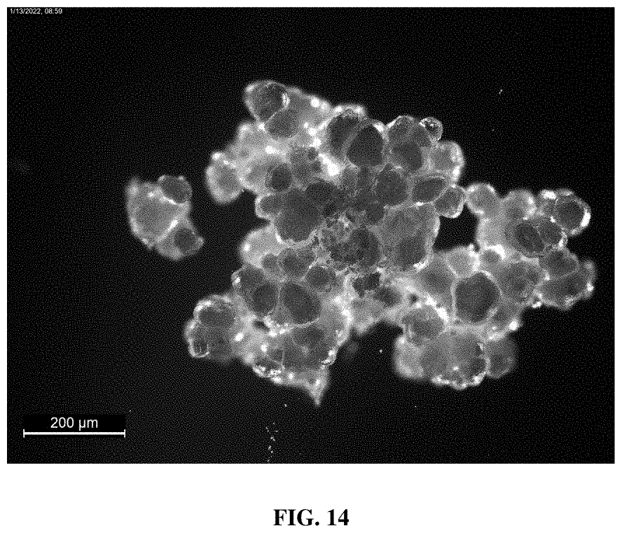

In another embodiment, NR—Cl was granulated in Isopropyl alcohol with ethyl cellulose, 4% fumaric acid and 1% stearic acid, resulting in a 10% weight gain to the NR—Cl. Then, an 87% Ethyl cellulose and 13% castor oil coating was applied utilizing a fluidized bed technology targeting a 100% weight gain. Then, a Beeswax coating was applied over the ethyl cellulose targeting an additional 10% weight gain. The final formulation had a theoretical % NR—Cl of 41.3% (w/w) and a particle size of approximately 50-100 μm in diameter (FIG. 14).

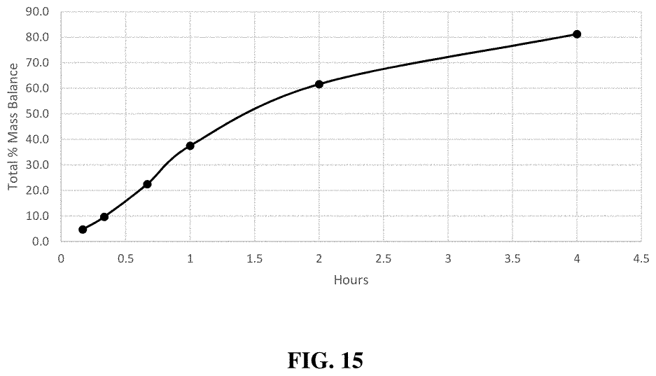

To evaluate the retention of NR—Cl, the formulation was added to an aqueous 10 mM phosphate solution, buffered at pH 3.0. The solution was analyzed for NR—Cl and its constituents to calculate mass balance recovery over four hours at ambient conditions with minimal agitation (Table 7 and FIG. 15). The 100% Ethyl Cellulose/10% Wax formulation released approximately 37.5% (w/w) of the NR—Cl in the formulation after 1 hour of stirring. Over the course of 4 hours, additional NR—Cl was released. At 4 hours, 81.2% (w/w) of the NR—Cl had been released.

TABLE 7

| | % Recovery |
|---|---|
| | (Mass |
| Hours | Balance) |
| 0.17 | 4.7 |
| 0.33 | 9.7 |
| 0.67 | 22.5 |
| 1.00 | 37.5 |
| 2.00 | 61.6 |
| 4.00 | 81.2 |

Table 7 shows total NR—Cl percent mass balance recovered from the 41.3% NR—Cl formulation in a 10 mM Phosphate buffered aqueous solution at a pH of 3.0 over four hours at ambient conditions.

FIG. 15 shows total NR—Cl percent mass balance released from the 41.3% NR—Cl formulation in a 10 mM phosphate buffered aqueous solution at pH 3.0 over four hours at ambient conditions.

The use of the terms “a,” “an,” “the,” and similar referents in the context of describing the presently claimed invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±10%; in other embodiments the values may range in value either above or below the stated value in a range of approximately ±5%; in other embodiments the values may range in value either above or below the stated value in a range of approximately ±2%; in other embodiments the values may range in value either above or below the stated value in a range of approximately ±1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entireties. The present invention may be embodied in other specific forms without departing from the spirit or essential attributes thereof and, accordingly, reference should be made to the appended claims, rather than to the foregoing specification, as indicating the scope of the invention.

We claim:

1. A composition comprising polymerized ethyl cellulose encapsulating nicotinamide riboside, or salts thereof, or solvates thereof, or derivatives thereof.

2. The composition of claim 1, wherein the nicotinamide riboside salt is a chloride salt (NR—Cl).

3. The composition of claim 1, wherein the nicotinamide riboside salt is selected from the group consisting of bromide, iodide, fluoride, formate, acetate, propionate, butyrate, glutamate, aspartate, ascorbate, benzoate, carbonate, citrate, carbamate, gluconate, lactate, methyl bromide, methyl sulfate, nitrate, phosphate, diphosphate, succinate, sulfate, tartrate, hydrogen tartrate, malate, hydrogen malate, maleate, fumarate, citrate, stearate, palmitate, myristate, laurate, caprate, caprylate, caproate, oleate, linoleate, sulfonate, trifluoromethanesulfonate, trichloromethanesulfonate, tribromomethanesulfonate, trichloroacetate, tribromoacetate, and trifluoroacetate.

4. The composition of claim 1, wherein the polymerized ethyl cellulose includes about 10% by weight additional nicotinamide riboside chloride (NR—Cl).

5. The composition of claim 1, wherein the polymerized ethyl cellulose is combined with a long chain fatty acid selected from the group consisting of saturated, unsaturated, and polyunsaturated fatty acids with carbon chain lengths of C₆-C₂₄.

6. The composition of claim 5, wherein the fatty acid is selected from the group consisting of stearic acid, palmitic acid, myristic acid, lauric acid, capric acid, caprylic acid, and caproic acid.

7. The composition of claim 1, wherein the polymerized ethyl cellulose is combined with a component selected from the group consisting of oxalic acid, malonic acid, succinic acid, glutaric acid, adipic acid, pimelic acid, suberic acid, fumaric acid, maleic acid, malic acid, tartaric acid, oxaloacetic acid, aspartic acid, glutamic acid, citric acid, methacrylate copolymer, polymethacrylate, chitosan, and zein protein.

8. The composition of claim 1, wherein the polymerized ethyl cellulose is combined with an edible oil or wax selected from the group consisting of food oil, vegetable oil, botanical oil, castor oil, palm oil, sunflower oil, carnauba wax, cottonseed oil, soybean oil, cocoa butter, paraffin wax, bees wax, high oleic safflower oil, soy oil, fractionated coconut oil, medium chain triglycerides, MCT oil, high oleic sunflower oil, corn oil, canola oil, coconut oil, palm kernel oil, marine oil, walnut oil, wheat germ oil, sesame oil, cod liver oil, candelilla wax, palm stearin, rapeseed oil, glycerol dibehenate, glycerol distearate, peanut oil, grape seed oil, sesame oil, borage oil, fish oil, sea buckthorne oil, flax oil, peanut oil, jojoba oil, corn oil, evening primrose oil, amaranth oil, safflower oil, soybean oil, palm oil, almond oil, cashew oil, hazelnut oil, macademia oil, pecan oil, pistachio oil, acai oil, blackcurrant oil, apricot oil, argan oil, artichoke oil, avocado oil, babassu oil, ben oil, borneo tallow nut oil, buffalo gourd oil, carob pod oil, coriander seed oil, false flax oil, hemp oil, kapok seed oil, lallemantia oil, meadowfoam seed oil, mustard oil, okra seed oil, perilla seed oil, pequi oil, pine nut oil, poppy seed, prune kernel oil, pumpkin seed oil, quinoa oil, ramtil oil, rice bran oil, tea oil, thistle oil, and mixtures thereof.

9. The composition of claim 1, wherein the amount of nicotinamide riboside, or a salt thereof, or a solvate thereof, is present in an amount of about 0.1% by weight to about 65% by weight of the encapsulated material in combination with one or more polymers.

10. A method of making a composition comprising polymerized ethyl cellulose encapsulating nicotinamide riboside, a salt thereof, or a solvate thereof, or a derivative thereof, comprising the steps of:
(a) dispersing nicotinamide riboside, a salt thereof, or a solvate thereof, or a derivative thereof, in a matrix of ethyl cellulose and a solvent to produce a suspension;
(b) homogenizing the suspension;
(c) atomizing the homogenized suspension to produce particles of polymerized ethyl cellulose encapsulating nicotinamide riboside, a salt thereof, or a solvate thereof; and
(d) drying the particles to provide ethyl cellulose coated particles of nicotinamide riboside, a salt thereof, or a solvate thereof, or a derivative thereof.

11. The method of claim 10, wherein the nicotinamide riboside salt is a chloride salt (NR—Cl).

12. The method of claim 10, wherein the nicotinamide riboside salt is selected from the group consisting of bromide, iodide, fluoride, formate, acetate, propionate, butyrate, glutamate, aspartate, ascorbate, benzoate, carbonate, citrate, carbamate, gluconate, lactate, methyl bromide, methyl sulfate, nitrate, phosphate, diphosphate, succinate, sulfate, tartrate, hydrogen tartrate, malate, hydrogen malate, maleate, fumarate, citrate, stearate, palmitate, myristate, laurate, caprate, caprylate, caproate, oleate, linoleate, sulfonate, trifluoromethanesulfonate, trichloromethanesulfonate, tribromomethanesulfonate, trichloroacetate, tribromoacetate, and trifluoroacetate.

13. A method of making a composition comprising polymerized ethyl cellulose encapsulating nicotinamide riboside, a salt thereof, or a solvate thereof, or a derivative thereof, comprising the steps of:
(a) combining nicotinamide riboside, a salt thereof, or a solvate thereof, or a derivative thereof, with at least one carboxylic acid selected from the group consisting of stearic acid, palmitic acid, myristic acid, lauric acid, capric acid, caprylic acid, caproic acid, oxalic acid, malonic acid, succinic acid, glutaric acid, adipic acid, pimelic acid, suberic acid, fumaric acid, maleic acid, malic acid, tartaric acid, oxaloacetic acid, aspartic acid, glutamic acid, and citric acid, to produce a mixture;
(b) granulating the combined mixture to produce particles;
(c) applying a coating of ethyl cellulose to the particles; and
(d) drying the particles to provide ethyl cellulose coated particles of nicotinamide riboside, a salt thereof, or a solvate thereof, or a derivative thereof.

14. The method of claim 13, wherein the nicotinamide riboside, a salt thereof, or a solvate thereof, or a derivative thereof, in step (a) is combined with ethyl cellulose and the at least one carboxylic acid.

15. The method of claim 13, wherein the granulating step (b) is performed with a solvent selected from the group consisting of acetone, methanol, ethanol, and isopropanol.

16. The method of claim 13, wherein the applying a coating step (c) is performed with an edible oil and the ethyl cellulose.

17. The method of claim 16, further comprising a step following step (c):
(c1) applying a coating of bees wax to the particles.

18. The method of claim 13, further comprising a step following step (c):
(c1) applying a coating of bees wax to the particles.

19. The method of claim 13, wherein the nicotinamide riboside salt is a chloride salt (NR—Cl).

20. The method of claim 13, wherein the nicotinamide riboside salt is selected from the group consisting of bromide, iodide, fluoride, formate, acetate, propionate, butyrate, glutamate, aspartate, ascorbate, benzoate, carbonate, citrate, carbamate, gluconate, lactate, methyl bromide, methyl sulfate, nitrate, phosphate, diphosphate, succinate, sulfate, tartrate, hydrogen tartrate, malate, hydrogen malate, maleate, fumarate, citrate, stearate, palmitate, myristate, laurate, caprate, caprylate, caproate, oleate, linoleate, sulfonate, trifluoromethanesulfonate, trichloromethanesulfonate, tribromomethanesulfonate, trichloroacetate, tribromoacetate, and trifluoroacetate.

21. The method of claim 13, wherein the amount of ethyl cellulose coating is in a range of about 30% by weight to about 100% by weight based on the combined mixture.

22. A composition comprising polymerized ethyl cellulose encapsulating a compound having the formula (Ia), or salts thereof, or solvates thereof: 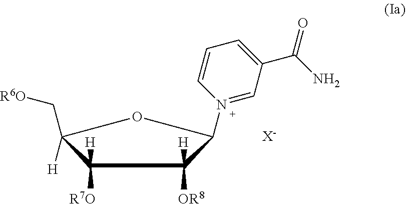
wherein R⁶ is selected from the group consisting of hydrogen, —C(O)R′, —C(O)OR′, —C(O)NHR′, substituted or unsubstituted (C₁-C₈)alkyl, substituted or unsubstituted (C₁-C₈)cycloalkyl, substituted or unsubstituted aryl, substituted or unsubstituted heteroaryl, and substituted or unsubstituted heterocycle;
wherein R′ is selected from the group consisting of hydrogen, —(C₁-C₈)alkyl, —(C₁-C₈)cycloalkyl, aryl, heteroaryl, heterocycle, aryl(C₁-C₄)alkyl, and heterocycle(C₁-C₄)alkyl;
R⁷ and R⁸ are independently selected from the group consisting of hydrogen, —C(O)R′, —C(O)OR′, —C(O)NHR′, substituted or unsubstituted (C₁-C₂₄)alkyl, substituted or unsubstituted (C₁-C₈)cycloalkyl, substituted or unsubstituted aryl, substituted or unsubstituted heteroaryl, substituted or unsubstituted heterocycle, substituted or unsubstituted aryl(C₁-C₄)alkyl, and substituted or unsubstituted heterocycle(C₁-C₄)alkyl; and
wherein X⁻ is selected from the group consisting of chloride, bromide, iodide, fluoride, formate, acetate, propionate, butyrate, glutamate, aspartate, ascorbate, benzoate, carbonate, citrate, carbamate, gluconate, lactate, methyl bromide, methyl sulfate, nitrate, phosphate, diphosphate, succinate, sulfate, tartrate, hydrogen tartrate, malate, hydrogen malate, maleate, fumarate, citrate, stearate, palmitate, myristate, laurate, caprate, caprylate, caproate, oleate, linoleate, sulfonate, trifluoromethanesulfonate, trichloromethanesulfonate, tribromomethanesulfonate, trichloroacetate, tribromoacetate, and trifluoroacetate.

23. The composition of claim 22, wherein for each of R⁶, R⁷, and R⁸ is —C(O)R′, R′ is methyl, and X⁻ is a chloride salt (NRTA-Cl).

24. The composition of claim 22, wherein for each of R⁶, R⁷, and R⁸ is —C(O)R′, R′ is methyl, and X⁻ is a salt selected from the group consisting of stearate, palmitate, myristate, laurate, caprate, caprylate, caproate, oleate, and linoleate.

25. The composition of claim 22, wherein the polymerized ethyl cellulose includes about 10% by weight additional of the compound of formula (Ia).

26. The composition of claim 22, wherein the polymerized ethyl cellulose is combined with a long chain fatty acid selected from the group consisting of saturated, unsaturated, and polyunsaturated fatty acids with carbon chain lengths of C₆-C₂₄.

27. The composition of claim 26, wherein the fatty acid is selected from the group consisting of stearic acid, palmitic acid, myristic acid, lauric acid, capric acid, caprylic acid, and caproic acid.

28. The composition of claim 22, wherein the polymerized ethyl cellulose is combined with a component selected from the group consisting of oxalic acid, malonic acid, succinic acid, glutaric acid, adipic acid, pimelic acid, suberic acid, fumaric acid, maleic acid, malic acid, tartaric acid, oxaloacetic acid, aspartic acid, glutamic acid, citric acid, methacrylate copolymer, polymethacrylate, chitosan, and zein protein.

29. The composition of claim 22, wherein the polymerized ethyl cellulose is combined with an edible oil or wax selected from the group consisting of food oil, vegetable oil, botanical oil, castor oil, palm oil, sunflower oil, carnauba wax, cottonseed oil, soybean oil, cocoa butter, paraffin wax, bees wax, high oleic safflower oil, soy oil, fractionated coconut oil, medium chain triglycerides, MCT oil, high oleic sunflower oil, corn oil, canola oil, coconut oil, palm kernel oil, marine oil, walnut oil, wheat germ oil, sesame oil, cod liver oil, candelilla wax, palm stearin, rapeseed oil, glycerol dibehenate, glycerol distearate, peanut oil, grape seed oil, sesame oil, borage oil, fish oil, sea buckthorne oil, flax oil, peanut oil, jojoba oil, corn oil, evening primrose oil, amaranth oil, safflower oil, soybean oil, palm oil, almond oil, cashew oil, hazelnut oil, macademia oil, pecan oil, pistachio oil, acai oil, blackcurrant oil, apricot oil, argan oil, artichoke oil, avocado oil, babassu oil, ben oil, borneo tallow nut oil, buffalo gourd oil, carob pod oil, coriander seed oil, false flax oil, hemp oil, kapok seed oil, lallemantia oil, meadowfoam seed oil, mustard oil, okra seed oil, perilla seed oil, pequi oil, pine nut oil, poppy seed, prune kernel oil, pumpkin seed oil, quinoa oil, ramtil oil, rice bran oil, tea oil, thistle oil, and mixtures thereof.

30. The composition of claim 22, wherein the amount of the compound having the formula (Ia), or a salt thereof, or a solvate thereof, is present in an amount of about 0.1% by weight to about 90% by weight of the encapsulated material in combination with one or more polymers.
